(19) United States
(12) Patent Application Publication
(10) Pub. No.: US 2016/0202250 A1
(43) Pub. Date: Jul. 14, 2016

(54) Chemiresistive Biosensor for the Quantitative Detection of Human Cardiac Biomarker and a Process Thereof

(71) Applicant: Council of Scientific & Industrial Research, New Delhi (IN)

(72) Inventors: Vikash SHARMA, New Delhi (IN); Vinod Kumar TANWAR, New Delhi (IN); Ashok Manikrao BIRADAR, New Delhi (IN); Rajesh, New Delhi (IN)

(73) Assignee: Council of Scientific & Industrial Research, New Delhi (IN)

(21) Appl. No.: 14/991,527

(22) Filed: Jan. 8, 2016

(30) Foreign Application Priority Data
Jan. 9, 2015 (IN) 0076/DEL/2015

(51) Int. Cl. G01N 33/543; C25D 1/00; C25D 1/18

(52) U.S. Cl. CPC G01N 33/5438; C25D 1/18; C25D 1/006; G01N 2333/47; G01N 2800/32; B82Y 5/00

(57) ABSTRACT

The present invention disclosed a metal nanoparticles/single-walled carbon nanotube (MNP/SWCNT) hybrid based chemiresistive biosensor for the quantitative detection of human cardiac biomarkers troponin I (cTnI) and myoglobin (Mb). The highly specific cardiac-antibody, anti-cTnI (Ab-cTnI) or anti-Mb (Ab-Mb), was covalently immobilized to site-specific carboxyl groups on MNP anchored over SWCNT device. The biosensor device was characterized by the source-drain current-voltage measurements. The device performance was investigated with a change in conductance in SWCNT channel upon exposure to cTnI in human serum. MNP provided large surface area for high protein loading and improved electrical signal by inducing charge density in SWCNT, resulting in low level detection of cTnI and Mb with high sensitivity.

### FIELD OF THE INVENTION

The present invention relates to a chemiresistive biosensor for the quantitative detection of human cardiac biomarkers and a process for the preparation thereof. Particularly, the present invention relates to a process for the preparation of a chemiresistive biosensor for the quantitative detection of human cardiac biomarkers such as troponin I and myoglobin. More particularly, the present invention also relates to biofunctionalize platinum nano particles decorated electrophoretically aligned single-walled carbon nanotube between a pair of gold microelectrodes to form a chemiresistive biosensor for the detection of human cardiac troponin I in normal human serum.

### BACKGROUND AND PRIOR ART OF THE INVENTION

Cardiovascular disease (CVD) is the leading cause of morbidity and mortality worldwide and accounts for approximately half of all the deaths within the western world. Coronary ischemia is the root cause of acute myocardial infarction (AMI) and hence its early and reliable detection is a prerequisite for appropriate triage decision in emergency room to initiate the right therapy. Generally, an electrocardiogram (ECG) is given to assess condition of heart; however 50-70% of patients experiencing CVD have normal or ambiguous ECG reading [Brogan G. X., Jr., Bock J. L. Clin Chem, 44, 1865-1859, 1998]. Cardiac markers continue to play a major role in the diagnosis and management of patients suspected of having myocardial damage or AMI. Previously, the commonly used biomarkers for early detection of CVD included the MB isoenzyme of creatine kinase (CK-MB) and myoglobin (Mb) [Adams J E, 3d, Bodor G S, Davila-Roman V G, Delmez J A, Apple F S, Ladenson J H, Jaffe A S., Circulation 88, 101-106, 1993]. However, in the recent years CK-MB has been replaced by cardiac troponin I (cTnI), a more specific biomarker. Troponin I is a subunit of cardiac troponin complex, which is broken up during the myocardial damage and the individual protein components are released in the blood stream [Fishbein M. C., Wang T, Matijasevic M, Hong L, Apple F. S. Cardivasc. Pathol. 12, 65-71, 2003]. The cTnI has high tissue specificity and virtually absent in skeletal muscle tissue. cTnI levels are measurable in serum within 4-6 h after the onset of AMI. The serum concentrations peaks at about 12 h, and remain diagnostic for at least 7 days post-AMI [Babuin L and Jaffe A. S. Can. Med. Assoc. J. 173(10) 1191-1202, 2005], hence provides a long window of detection of cardiac injury allowing for reliable point-of-care detection. Therefore, cTnI in the human serum has been considered as the “gold standard” for diagnosis of myocardial injury[Antman E., Bassand J-P, Klein W., Ohman M., Sendon J. L. L., Rydén L., Simoons M., Tendera M. J. Am. Coll. Cardiol. 36, 959-969, 2000]. The National academy of Clinical Biochemistry (NACB), USA and International Federation of Clinical Chemistry (IFCC), Germany have recommended the use of two decision cut off limits for cardiac Troponins. A low limit for establishing myocardial injury and a high limit that qualifies as AMI. Based on literature data and clinical assessments cTnI levels greater than 0.1 ng/ml places a patient with unstable angina in the high-risk category for short-term risk of death or non-fatal MI. A cut-off value of greater than 1.2 ng/ml is taken as the definition of AMI, making a cut off value of 0.1 ng/ml of cTnI to identify patients at higher risk for very early adverse outcomes.

Therefore, there is an urgent need to develop a point-of-care device for rapid and quantitative detection of cardiac biomarker cTnI at least 0.1 ng/ml detection limit with high sensitivity (output signal) by the unskilled/semi-skilled person for the cost-effective diagnosis of AMI and for effective management of patients, resulting in major cost benefit to the health service sector.

Cardiac myoglobin, a heme-containing protein, although not a very specific marker, but it is the first marker released after the myocardial damage (as early as 1-3 h upon symptom onset), due to its small size (17.8 kDa) makes it highly sensitive and valuable marker for the early diagnosis of AMI [Lin H., Rick J., Chou T., Biosensor and. Bioelectron. 22, 3293-3301, 2007; Moreira F. T. C., Dutra R. A. F., Noronha J. P. C., Sales M. G. F., Biosens. Bioelectron. 26, 4760-4766, 2011 and Jaffe A. S., Babuin L., Apple F. S., JACC 48, 1-11, 2006]. The normal Mb-level in the blood ranges from 30-90 ng mL⁻¹ which rise up to 900 ng mL⁻¹ after AMI. The cut-off concentration of myoglobin in the blood is 70-200 ng mL⁻¹[Qureshi A., Gurbuz Y., Niazi J. H., Sensors and actuators B 171, 62-76, 2012]. The traditional detection methods for the estimation of cardiac biomarkers are sandwich immunoassay with secondary labeled antibodies, enzyme-linked immunosorbent assay (ELISA) and fluorescence. However, these methods have many disadvantages such as time consuming, complicated multistage process and difficulty to realize automation with the aim of rapid screening [Grachev M. A., Matvev L. E., Pressman E. K., Roschke V. V., Clin. Chim. Acta 124, 235, 1982; Olsson T., Bergstrom K., Thore A., Clin. Chim. Acta 138, 31, 1984] and therefore, demand for more sensitive and rapid technology platform for the diagnosis of cardiovascular disease.

In recent years, there has been escalating interest in the use of one dimensional nanostructures including nanowires, nanobelts, and nanotubes as attractive transducer platform in the fabrication of chemiresistive biosensor [Wanekaya, A. K., Chen, W., Myung, N. V., Mulchandani, A., Electroanalysis, 18, 533-550, 2006]. Single-walled carbon nanotubes (SWCNTs) are one such class of nanomaterials, which have been extensively used as transducing element [Mao S., Lu G., Yu K., Chen J., Carbon, 48, 479-486, 2010] because of their excellent mechanical and electronic properties [Iijima S., Nature 354, 56-58,1991; Treacy M. M. J., Ebessen T. W., Gibson J. M., Nature 381, 678-680,1996], high surface to volume ratio. These are extremely sensitive to electronic perturbations on account of the surrounding environment, as the electric current flows through the uppermost layer of SWCNT and are in direct contact with the analyte of interest [Cella L. N., Chen W., Myung N. V., and Mulchandani A., J. A. C. S, 132, 5024-5026, 2010]. In SWCNTs based FET (field effect transistor) sensors, the proposed mechanisms include charge-induced electrostatic gating, Schottky barrier effect, capacitance modulation, and carrier mobility change [Chang J., Mao S., Zhang Y., Cui S., Steeber D. A., Chen J., Biosensors and Bioelectronics 42, 186-192, 2013]. These FET/chemiresistive biosensors have several advantages over the conventional biosensing techniques with features of (i) a small sample volume requirement, (ii) label free detection (iii) high sensitivity and specificity, (iv) portable, disposable and low cost. [Kim J. P., Lee B. Y., Lee J., Hong S., Sim S. J., Biosensors and Bioelectronics 24, 3372-3378, 2009; Das B. K., Tlili C., Badhulika S., Cella L. N., Chen W. and Mulchandani A., Chem. Commun., 47, 3793-3795, 2011].

US2013/0337567 a nanowire field effect transistor biosensor with improved sensitivity. The said invention particularly disclosed a silicon nanowire FET biosensor for a low level detection of biomolecule. However, the disadvantages of the fabricated FET biosensor include the use of complicated multiple nanowire connected to a source, drain and base electrodes and is not cost effective for mass production.

US2012/0214172 a graphene based field effect transistor biosensor which is a FET based bio sensor using metal nitride/graphene hybrid sheet as a conducting channel. However, this invention has a disadvantage of using a drop cast method for the immobilization of bioconjugate, which is prone to unstable protein binding to the conducting channel due to the weak electrostatic interaction.

US20140162390 and US 20140162375 discloses a carbon based biosensors and processes for the manufacture thereof. The disclosed carbon based biosensor includes a channel surface formed of graphene or carbon nanotubes (CNT) functionalized with an imidazolidone compound. The imidazolidone compound includes an imidazolidone ring for self assembly to the carbon surface, e.g., graphene or CNT, with an at least one additional functionality, for the selective immobilization of a targeted analyte. The novelty in the present invention lies in the use of imidazolidone ring, acting as a crosslinker for the stable immobilization of biomolecules. However, these imidazolidone ring based compounds are very expensive and required additional resources for chemical synthesis and its use is not advantageous for biosensor fabrication.

US 20120073992 relates to a biosensor based on a carbon nanotube-electric field effect transistor. More particularly, the invention relates to a conducting carbon nanotube channel, which is discontinued in the middle, wherein a receptor is fixed on for detecting a target biomaterial. This discontinued area is deposited with a metal such as gold for the immobilization of biomolecular probe. The rest of the area of the device is covered by insulating layer of polydimethyl siloxane (PDMS) and Teflon. This biosensor device suffers from disadvantages of expensive, tedious and time consuming steps of surface modification that includes cutting of carbon nanotube using laser ablation to form a discontinuous distance of 10-2000 micro meter (detection zone), deposition of gold layer to bridge the discontinuous distance, and insulating layer to prevent the source and drain electrodes from interaction with biomolecules.

Recently we have reported (Applied Physics Letters 103, 203703(2013)) protein antibody functionalized gold nano particles (GNP) decorated carbon nano tube (GNP/SWCNT) hybrid as a conducting channel between the gold electrodes for the detection of cTnI in phosphate buffer (PBS). This device could detect cTnI with a linear response (current/resistance) to cTnI over the concentration range of 0.01 ng/ml to 10 ng/ml in phosphate buffer solution (PBS). However, it had a disadvantage of not detecting cTnI with a linear response in current/resistance below 0.01 ng/ml cTnI concentration in PBS. In addition to this the sensing performance of the device was not so adequate for cTnI detection in human serum (not reported in the cited reference) as human serum is more viscous than PBS that hindered the ionic movements on sensing region/zone, a fact that has been reported earlier in the literature [A carbon nanotube metal semiconductor field effect transistor-based bio sensor for detection of amyloid-beta in human serum; Jeseung Oh, et al.; Biosensors and Bioelectronics 50(2013) 345-350]. Its sensing performance has been compared with that of the Pt(MPA)/SWCNT NP hybrid based chemiresistive bio sensor towards cTnI concentration in human serum in the present invention (Example 11 of the present invention). This disadvantage was overcome by using metal nano particles having high work function [(Φ)>4.3-4.9 eV] such as platinum nano particles (PtNP) with a work function (Φ) of 6.4 eV functionalized over SWCNT for making a conducting channel in the biosensing device for low level concentration detection of cTnI, as disclosed in the present patent proposal. The use of high work function of Pt NP facilitated the transfer of electrons from SWCNT to Pt NP that enhances the current signals in the p type SWCNT conducting channel and has been shown experimentally in present invention (see Example 9 and FIG. 3).

Innam Lee et. al. published (Biosensors 2: 2012, 205-220) in article titled “Detection of Cardiac Biomarkers Using Single Polyaniline Nanowire-Based Conductometric Biosensors”, wherein the polyaniline (PANI) nanowire bio sensors were developed that can detect cardiac biomarkers such as Myo, cTnI, CK-MB. Though the device is capable of detecting a low level cTnI concentration up to as low as 250 fg/ml in PBS, but the electrical current signal is too low with change in the current (ΔG/G₀˜0.01) of 1% only for 500 fg/ml in PBS [FIG. 3(a) in the cited reference]. Further, a current response of 5% was obtained for the detection of 30 pg/ml cTnI, (when 100 μg/ml protein antibody, cTnI mAbs, was used for functionalization of PANI) [line 8-10, para 3, page 213 of the cited references]. The device provided a current response from 2% to 18% (AG/G₀=0.02-0.18) for the concentration range of 250 fg/ml to 3 ng/ml cTnI in PBS [FIG. 4(b) in the cited reference]. Such a low current signal amounts to electrical noise that pertains to fluctuations in the detection of the target molecules. Even such a low electrical signal output (2%-18%) was obtained after using high concentration of protein antibody (cTnI mAbs) of 200 μg/ml for the functionalization of PANI in the device fabrication. Since the protein antibody is quite expensive, the high concentration of protein antibody used in the disclosed device does not makes it a cost effective biosensor.

Reference may be made to Wei-Wen Liu et. al. 2012. IEEE EMBS International Conference on Biomedical Engineering and Sciences) wherein the CNT based FET devices are reported for detection of biomolecules. However these CNT based FET devices are not sufficiently good for low level detection of biomolecules.

Another reference may be made to Tao Kong et. al. (2012. Biosensors Bioelectronics, Vol. 34(1) 267-272) describing “A label free biosensor for electrical detection of cardiac troponin I (cTnI) using silicon nanowire (SiNW) based field effect transistor (FET). This device provided a lowest detection of 0.092 ng/ml cTnI, with a sensitivity of 6.4% change in the current for the detection of 0.23 ng/ml cTnI in PBS. This sensitivity is not sufficiently high for low level cTnI detection without acceptable signal noise. Apart from this the device involved multiple steps of fabrication including wet etching, that makes it a time consuming and quite expensive process.

Though FET/chemiresistive bio sensors have been reported elsewhere for low level molecular detection, but no one has yet reported a sensitive chemiresistive biosensor using Platinum nanoparticles/SWCNT hybrid as a conductance material for human cardiac biomarker detection in human serum. To the best of our knowledge based on available literature, this is the first chemiresistive biosensor based on Pt/SWCNT hybrid based for the quantitative detection of human cardiac biomarker such as cTnI and Mb. The bio sensor fabrication and the sensing performance were investigated by the measurement of current-voltage (I-V) and FET transfer characteristics.

### OBJECTIVES OF THE INVENTION

Main object of the present invention is to provide a chemiresistive bio sensor for the quantitative detection of human cardiac biomarkers troponin I and myoglobin.

Another object of the present invention is to provide a process for the preparation of a chemiresistive biosensor for the quantitative detection of human cardiac biomarkers troponin I and myoglobin.

Yet another object is to provide a process wherein functionalized metal nanoparticles decorated single-walled carbon nanotube are electrophoretically aligned between a pair of gold microelectrode to form a SWCNT-chemiresistive device.

Yet another object is to provide a process wherein the metal nanoparticles used for making MNPs/SWCNT is Pt.

Yet another object is to provide a process wherein the functionalized metal nanoparticles used is selected from the group consisting of Mercaptopropionic acid (MPA) capped platinum nanoparticles, Pt(MPA)NP.

Yet another object is to provide a process wherein the functionalized metal nanoparticles are covalently immobilized with protein antibody specific to target biomolecules (antigen).

Yet another object is to provide a process wherein a small sample volume of the target biomarker antigen is required for detection

Yet another object is to provide a process wherein the target cardiac biomarker is quantified by measuring the change in current in the MNPs/SWCNT channel between the gold microelectrodes in a device, due to antibody-antigen interaction, as function of target cTnI or Mb concentration.

### SUMMARY OF THE INVENTION

Accordingly, the present invention provides A label free chemiresistive biosensor device for quantitative detection of human cardiac biomarker, wherein cardiac biomarkers are selected from human cardiac troponin I (cTnI) and myoglobin (Mb), said biosensor device comprising aligned carbon nanotubes (CNT) channel bridging an interspaced gap between microfabricated gold source and drain electrode over silicon dioxide coated silicon wafer having gold electrode surface passivated with thiol molecules and the CNT channel being functionalized with platinum nanoparticles through a bilinker 1-pyrenemethylamine hydrochloride and said platinum nanoparticles being covalently immobilized with cardiac biomarker specific protein antibody anti-cTnI (Ab-cTnI) or anti-Mb (Ab-Mb) and blocking non-specific binding sites with a blocking reagent.

In another embodiment of the present invention the carbon nanotube is a single-walled carbon nanotube (SWCNTs) having 80% p-type semiconducting characteristics.

In yet another embodiment of the present invention the interspaced gap between microfabricated gold source and drain electrodes is in the range of 1-5 μm.

In yet another embodiment of the present invention the platinum nanoparticles is capped with molecules having functional groups selected from the group consisting of carboxyl, amino and hydroxyl.

In yet another embodiment of the present invention the platinum nanoparticles is capped with mercaptopropionic acid.

In yet another embodiment of the present invention the size of the platinum nanoparticles is in the range of 2-20 nm.

In yet another embodiment of the present invention the size of the platinum nanoparticles is in the range of 2-10 nm.

In yet another embodiment of the present invention the size of the platinum nanoparticles used is preferable in the range of 2-5 nm.

In yet another embodiment of the present invention the cardiac biomarker specific protein antibody used is selected from anti-cTnI (Ab-cTnI) and anti-Mb (Ab-Mb).

In yet another embodiment of the present invention the cardiac biomarker used is selected from cTnI and Mb.

In yet another embodiment of the present invention the biosensor device is capable of detecting human cardiac cTnI in the range of 0.001 ng to 10.0 ng mL⁻¹ in normal human serum.

In yet another embodiment of the present invention the biosensor device is selective and sensitive to cTnI with a sensitivity of about 11% to 75% change in resistance (or current) over the concentration range of 0.001 ng to 10.0 ng mL⁻¹ cTnI in human serum.

In yet another embodiment of the present invention the device is capable of detecting myoglobin in the range of 0.03 ng to 1000 ng mL⁻¹ in phosphate buffer saline.

In yet another embodiment of the present invention the device provided a lowest detection limit of 0.03 ng mL⁻¹ for myoglobin.

In yet another embodiment of the present invention the device is selective and sensitive to myoglobin with a sensitivity of about 23% change in resistance per decade of myoglobin.

In yet another embodiment of the thiol molecules are selected from the group consisting of 6-mercapto-1-haxanol and ethane hexanol.

In yet another embodiment of the blocking reagent is selected from the group consisting of bovine serum albumin, ethylamine and Polyoxyethylene (20) sorbitan monolaurate.

The present invention further provides a process for the preparation of a label free chemiresistive bio sensor comprising the steps of:
i) bridging interspaced gap in between microfabricated gold electrode prepared over the silicon dioxide coated silicon substrate by electrophoretically aligning the single walled-carbon nanotubes, followed by annealing at a temperature of 200-400° C. under reduced environment of a gaseous mixture of 5-10% hydrogen in nitrogen gas, washing with distilled water and drying under N₂ gas flow to obtain a aligned single walled carbon nanotubes device (SWCNTs),
ii) treating the aligned single walled carbon nanotubes, as obtained in step (i) with a bilinker 1-pyrenemethylamine hydrochloride having terminal amino group in solvent for a period of 1-3 h and passivating gold source and drain electrodes by treating the single walled with thiol molecule in dimethyl formamide for a period of 1-5 h, followed by washing successively with dimethyl formamide and distilled water and dried under N₂ gas flow to obtain bilinker modified single walled-carbon nanotubes,
iii) functionalizing the bilinker modified single walled-carbon nanotubes, as obtained in step (ii) with carboxyl capped platinum nanoparticles (PtNP) by exposing the modified SWCNT-device to an aqueous solution of metal nanoparticles containing N-(3-dimethylaminopropyl)-N-ethyl carbodiimide hydrochloride and N-hydroxy succinimide for 1-5 h, rinsing thoroughly with double distilled water and drying under N₂ gas flow to obtain the PtNPs/SWCNT hybrid device,
iv) immobilizing the cardiac biomarker specific protein antibody on PtNPs/SWCNT as obtained in step (iii) by exposing the hybrid device to antibody solution in phosphate buffer saline, at pH 7.0-7.6, at a temperature of 4-10° C., for a period of 10-24 h, followed by washing with double distilled water and drying under N₂ gas flow and passivating the device with a blocking reagent to obtain the chemiresistive bio sensor device.

In yet another embodiment of the present process the interspaced gap in between the microfabricated gold source and drain electrode is in the range of 1-10 p.m.

In yet another embodiment of the present process the carbon nanotubes are electrophoretically aligned at an ac frequency of 4-10 MHz.

In yet another embodiment of the present process the platinum nanoparticles used is capped with molecules having functional groups such as carboxyl, amino or hydroxyl.

In yet another embodiment of the present process the carboxyl capped platinum nanoparticles used is mercaptopropionic acid capped metal nanoparticles.

In yet another embodiment of the present process, the size of the platinum nanoparticles used is in the range of 2-10 nm.

In yet another embodiment of the present process the protein antibody used is selected from anti-cTnI (Ab-cTnI) and anti-Mb (Ab-Mb).

In yet another embodiment of the present process the cardiac specific biomarker is selected from cTnI and Mb.

In yet another embodiment of the present process the device obtained is capable of detecting human cardiac cTnI in the range of 0.001 ng to 10.0 ng mL⁻¹ in normal human serum.

In yet another embodiment of the present process the device obtained is selective and sensitive to cTnI with a sensitivity of about 11% to 75% change in resistance over the concentration range of 0.001 ng to 10.0 ng mL⁻¹ cTnI in human serum.

In yet another embodiment of the present process the device obtained is capable of detecting myoglobin in the range of 0.03 ng to 1000 ng mL⁻¹ in phosphate buffer saline.

In yet another embodiment of the present process the device provides a lowest detection limit of 0.03 ng mL⁻¹ for myoglobin.

Novelty of the present invention lies in the development of novel miniaturized carboxyl functionalized Pt NP/SWCNT hybrid conducting channel based chemiresistive sensor for the label free quantitative detection of cardiac biomarkers such as cTnI and Mb.

The inventive steps lies in the use of carboxyl capped platinum nanoparticles of size ranging between 2-5 nm anchored on single-walled carbon nanotube that provided large surface area for high protein antibody loading and improved electrical signal by inducing charge density in SWCNT, resulting in low level detection of cardiac biomarkers cTnI and Mb with a linear response from 0.001 ng mL⁻¹ to 10 ng mL⁻¹ cTnI and 0.1 ng mL⁻¹ to 1000 ng mL⁻¹ Mb with high sensitivity (change in electrical signal).

### BRIEF DESCRIPTION OF THE DRAWINGS/FIGURES

FIG. 1 represents the current versus voltage (I-V) curves of the chemiresistive biosensor device at different stages of fabrication for the detection of cTnI

FIG. 2 represents the current versus voltage (I-V) curves of the chemiresistive biosensor device at different stages of fabrication

FIG. 3 represents the current versus voltage (I-V) curves with a change in conductance (Δ G/G₀) of: (a) SWCNT/PyMeNH₂/MCH/Pt (MPA) NP chemiresistive device with respect to SWCNT/PyMeNH₂/MCH and (b) SWCNT/PyMeNH₂/MCH/Au (MPA) NP chemiresistive device with respect to SWCNT/PyMeNH₂/MCH.

FIG. 4 represents the sensing performance of the SWCNT/PyMeNH₂/MCH/Pt(MPA)NP chemiresistive devices with current versus voltage (I-V) curves at different concentration of cTnI from 0.001 ng mL⁻¹ to 10 ng mL⁻¹ in human serum.

FIG. 5 represents the concentration dependent calibration curve showing a comparative sensing performance of the SWCNT/PyMeNH₂/MCH/Pt(MPA)NP ( . . . ) and SWCNT/PyMeNH₂/MCH/Au(MPA)NP (-) chemiresistive devices with current versus voltage (I-V) curves at different concentration of cTnI from 0.001 ng mL⁻¹ to 10 ng mL⁻¹ in human serum. Each data point is an average of the measurements from 3 independent sensors prepared at different time and error bars represent ±1 standard deviation; insets shows the device sensing response for non-specific cardiac C-reactive protein, human cardiac myoglobin and mouse IgG with respect to specific cardiac biomarker cTnI.

FIG. 6 represents the concentration dependent calibration curve of the chemiresistive bio sensor device for myoglobin detection. Each data point is an average of the measurements from 3 independent sensors prepared at different time and error bars represent ±1 standard deviation.

### DETAILED DESCRIPTION OF THE INVENTION

The present invention described the fabrication of Pt(MPA)/SWCNTs hybrid based nanoelectronic chemiresistive sensor for the quantitative detection of cardiac biomarkers cTnI and Mb. SWCNTs were aligned in parallel bridging gold electrodes by dielectrophoresis method to function as a conducting channel. The MPA capped Pt NPs were attached to SWCNT through an organic molecular bilinker 1-pyrenemethylamine. The MPA capped Pt NPs provided the pendant carboxyl functional groups for the covalent immobilization of protein antibody, anti-cTnI (Ab-cTnI) or anti-Mb (Ab-Mb), through carbodiimide coupling reaction. High protein loading with efficient covalent bonding to Pt(MPA)/SWCNTs nanocomposite resulted in wider linear detection range, high sensitivity and selectivity. The fabrication of sensor and its performance along with sensing mechanism were investigated by the measurement of I-V and FET transfer characteristics.

### EXAMPLES

Following examples are given by way of illustration therefore should not be construed to limit the scope of the invention.

Materials and Reagents used:

Ab-cTnI (Cat 4T21 MAb 19C7), human cardiac troponin I, cTnI (Cat 8T53), Ab-Mb (Cat 4M23 MAb 4E2), and Mb (Cat 8M50), were purchased from Hytest (Turku, Finland). Mouse IgG was purchased from Bangalore Genei, India

### Example 1

### SWCNT-Device Fabrication

A SWCNT suspension was prepared by suspending 0.1 mg SWCNT in 10 mL N, N-dimethylformamide (0.01 mg mL-1) followed by 90 min sonication. The above solution was centrifuged at 12,000 rpm for 90 min and the supernatant was collected. 1.0 μL drop of the above prepared SWCNT solution was pipetted out in between the 3 μm apart gold electrodes and then aligned by ac dielectrophoresis by applying an AC frequency of 4 MHz with 1.5V peak-to-peak amplitude until a resistance of 1 M Ω was obtained. The aligned SWCNTs were then annealed in inert flow environment (95% N2 and 5% H2) for 1 h at 300° C. to remove residual solvents and improve the contact between SWCNTs and electrodes.

### Example 2

The SWCNT-device fabrication was carried out under identical experimental conditions, as described in example 1, except applying an AC frequency of 5 MHz with 2.0V peak-to-peak amplitude until a resistance of 0.8 M Ω was obtained.

### Example 3

The SWCNT-device fabrication was carried out under identical experimental conditions, as described in example 1, except applying an AC frequency of 10 MHz with 3.0V peak-to-peak amplitude until a resistance of 0.5 M Ω was obtained.

### Example 4

### Functionalization of Carbon Nanotube (SWCNTs) with Carboxyl Capped Metal Nanoparticles

The aligned SWCNTs device was modified with bilinker, 1-pyrenemethylamine hydrochloride (PyMe-NH₂) by incubating it with 6 mM PyMe-NH₂ in DMF, for 2 h, followed by extensive washing with DMF and then dried under N₂ gas flow. The nonspecific binding sites at gold microelectrodes surface was then passivated by incubating the device with 6 mM 6-mercapto-1-haxanol (MCH) in DMF for 1 h. The PyMe-NH₂ functionalized aligned SWCNT device was incubated with 0.1 mg mL⁻¹ aqueous solution of mercaptopropionic acid (MPA) capped platinum nanoparticles [Pt(MPA)] of 2-5 nm size distribution containing 0.15 M N-(3-dimethylaminopropyl)-N-ethyl carbodiimide hydrochloride (EDC) and 0.03 M N-hydroxy succinimide (NHS) for 3 h, rinsed thoroughly with double distilled water and dried under N₂ gas flow to obtain the Pt(MPA)/SWCNT hybrid device.

### Example 5

The functionalization of SWCNT device was carried out with Pt (MPA) NPs by following the procedure as described in Example 4, except 10 mM PyMe-NH₂ in DMF was used, as bilinker, with an incubation time of 1 hr and the gold microelectrodes surface was passivated on treatment with 8 mM 6-mercapto-1-haxanol (MCH) in DMF for 30 minutes. The SWCNT device was treated with 0.2 mg mL⁻¹ aqueous solution of mercaptopropionic acid (MPA) capped platinum nanoparticles [Pt(MPA)], for a time period of 2 h, followed by extensive washing with DMF and then dried under N₂ gas flow to obtain the Pt (MPA)/SWCNT device.

### Example 6

Biomolecular Immobilization of Pt (MPA)/SWCNT Device with Protein Antibody for the Preparation of Chemiresistive Biosensor

The Pt (MPA)/SWCNT device of was incubated with 10 μL volume of 100 μg mL⁻¹ anti-cTnI (Ab-cTnI) concentration in phosphate buffer saline (PBS; pH 7.4) at 4° C. for a period of 6 h. This was followed by washing with PBS and double distilled water and dried under N₂ gas flow to obtain the Ab-cTnI-Pt (MPA)/SWCNT biosensor device. The device was further treated with 0.01% (w/v) bovine serum albumin (BSA) for a period of 1 h to block the non-specific sites of the unbound free NHS activated carboxyl groups on Pt(MPA) NPs and the residual SWCNT surface.

### Example 7

The Pt (MPA)/SWCNT device of was incubated with 5 μL volume of 100 μg mL⁻¹ anti-cTnI concentration in phosphate buffer saline (PBS; pH 7.4) at 4° C. for a period of 12 h. This was followed by washing with PBS and double distilled water and dried under N₂ gas flow to obtain the Ab-cTnI-Pt (MPA)/SWCNT chemiresistive biosensor. The device was further treated with 0.1% (w/v) bovine serum albumin (BSA) for a period of 30 min to block the non-specific sites of the unbound free NHS activated carboxyl groups on Pt(MPA) NPs and the residual SWCNT surface. The detail IV characteristics have been shown in FIG. 1.

### Example 8

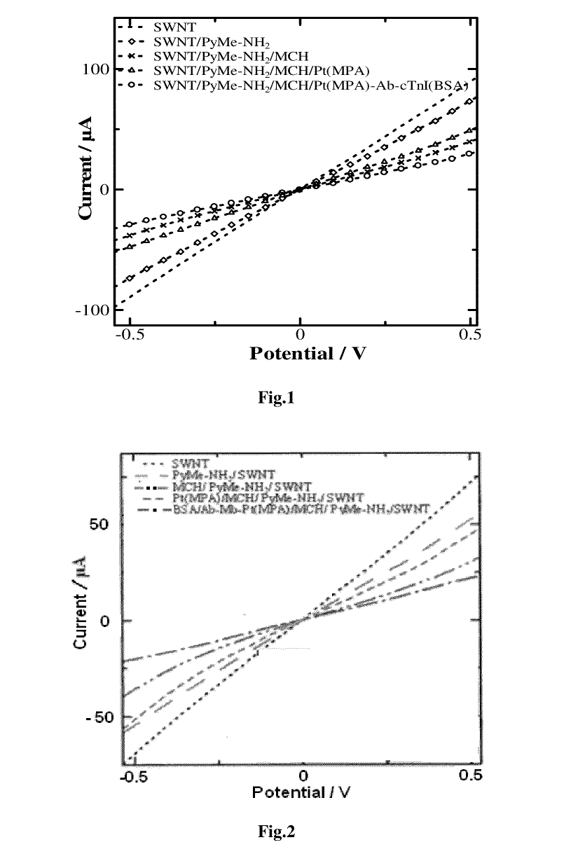

The Pt (MPA)/SWCNT device of was incubated with 5 μL volume of 100 μg mL-1 anti-Mb concentration (Ab-Mb) in phosphate buffer saline (PBS; pH 7.4) at 4° C. for a period of 12 h. This was followed by washing with PBS and double distilled water and dried under N2 gas flow to obtain the Ab-Mb-Pt (MPA)/SWCNT chemiresistive biosensor. The device was further treated with 0.1% (w/v) bovine serum albumin (BSA) for a period of 30 min to block the non-specific sites of the unbound free NHS activated carboxyl groups on Pt(MPA) NPs and the residual SWCNT surface (FIG. 2).

### Example 9

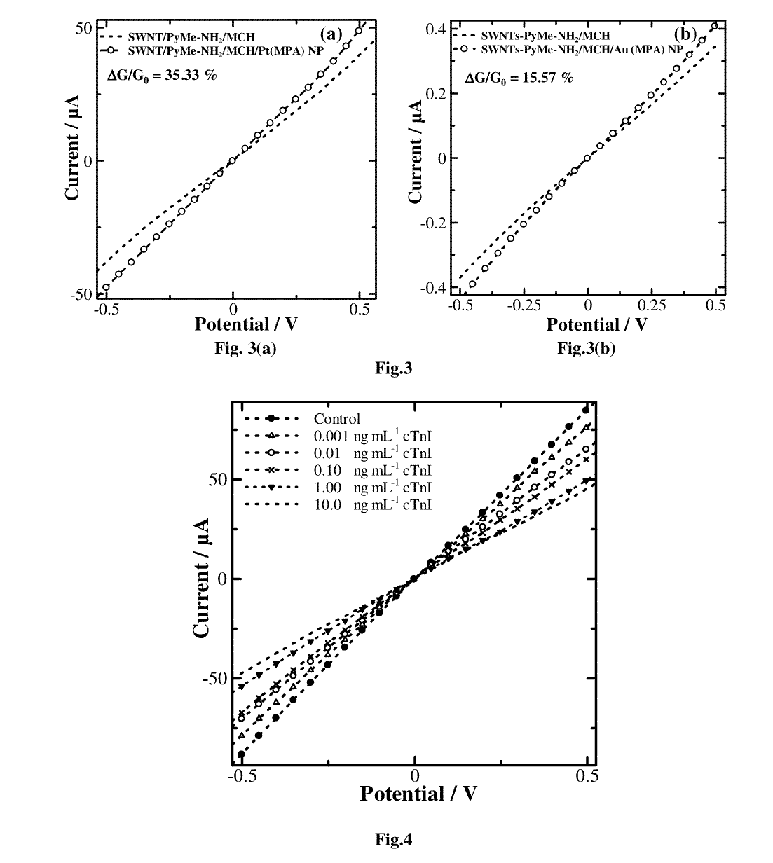

I-V measurements were conducted for investigating the comparative conductance performance (Δ G/G₀) between the devices: (a) SWCNT/PyMeNH₂/MCH/Pt (MPA) NP and (b) SWCNT/PyMeNH₂/MCH/Au (MPA) NP chemiresistive device with respect to SWCNT/PyMeNH₂/MCH. It has been found that the SWCNT/Pt NP device showed a high output conductance of A G/G₀=35.3% (FIG. 3a) in comparison to the SWCNT/AuNP with Δ G/G₀=15.5% (FIG. 3b).

### Example 10

### Sensing Performance of the Chemiresistive Biosensor for Human Cardiac Troponin I in Human Serum

The sensing performance of the Ab-cTnI-Pt(MPA)/SWCNTs and hybrid device was investigated by exposing it to the different concentrations of human cardiac cTnI in human serum. A calibration curve was plotted between the normalized resistance change [(R−R₀)/R₀, where R₀ and R is the resistance of the device measured before and after exposure to human cardiac cTnI antigen (Ag-cTnI) in human serum, respectively] and logarithmic concentration of the target cTnI. The resistance of the device was calculated by taking the reciprocal of the slope of the I-V plot between −0.5 and 0.5 V. The device was incubated with each 2 μL aliquot of different concentrations of cTnI i.e. 0.001 ng mL⁻¹, 0.01 ng mL⁻¹, 0.1 ng mL⁻¹, 1.0 ng mL⁻¹ and 10.0 ng mL⁻¹ at an interval of 1 min, at 25° C. after rinsed thoroughly with double distilled water and dried under N₂ for each successive I-V characteristic measurements (FIG. 4). The device exhibited a linear response to the target human cardiac cTnI antigen in human serum, over the concentration range of 0.001 ng mL⁻¹ to 10 ng mL⁻¹ with sensitivity (slope of the calibration curve) of about 15% per decade (FIG. 5).

### Example 11

### Experiments for a Comparative Sensing Performance Studies Between Pt NP/SWCNT and AuNP/SWCNT Hybrid Channel Based Chemiresistive Biosensor for Human Cardiac Troponin I in Human Serum

The sensing performance of the SWCNT/PyMeNH₂/MCH/Pt(MPA)NP device was compared with SWCNT/PyMeNH₂/MCH/Au(MPA)NP by measuring the resistance from current versus voltage (I-V) characteristic curves at different incubated concentration of cTnI in human serum and the corresponding calibration curve is shown in FIG. 5. SWCNT/PyMeNH₂/MCH/Au(MPA)NP could detect cTnI linearly over a concentration range of 0.01 ng mL⁻¹ to 10 ng mL⁻¹, which is a comparatively lower range of cTnI detection in comparison to a linear range of cTnI detection (0.001 ng mL⁻¹ to 10 ng mL⁻¹) obtained from SWCNT/PyMeNH₂/MCH/Pt(MPA)NP device with a sensitivity of 12% per decade in human serum. The SWCNT/PyMeNH₂/MCH/Pt(MPA)NP-Ab-cTnI(BSA) device was found to be more sensitive to cTnI than SWCNT/PyMeNH₂/MCH/Au(MPA)NP-Ab-cTnI(BSA) device in human serum, especially in a low concentration side, with an increment of the order of 10 in human serum.

### Example 12

### Inter and Intra Device Sensing Performance Towards the Target cTnI

The device sensing performance was investigated with three different chemiresistive devices prepared at different time. The results showed 9.8% inter-device relative standard deviation (RSD) in resistance upon exposure to a fixed concentration of 1.0 ng mL⁻¹ cTnI in human serum.

### Example 13

### Selectivity of Device Towards Target cTnI

Selectivity of device towards the target cardiac cTnI, was investigated by exposing it with other cardiac specific biomarkers i.e. 1.0 ng mL⁻¹C-reactive protein (CRP), 1.0 ng mL⁻¹ Mb and 1.0 ng mL⁻¹ non-specific protein antigen (mouse Immunoglobulin; IgG). The normalized response (%) of the device for CRP, Mb and IgG with respect to target cTnI was found to be 11.8%, 9.23 and 11.0%, respectively (Inset of FIG. 5).

### Example 14

### Sensing Performance of the Chemiresistive Biosensor for Human Cardiac Myoglobin

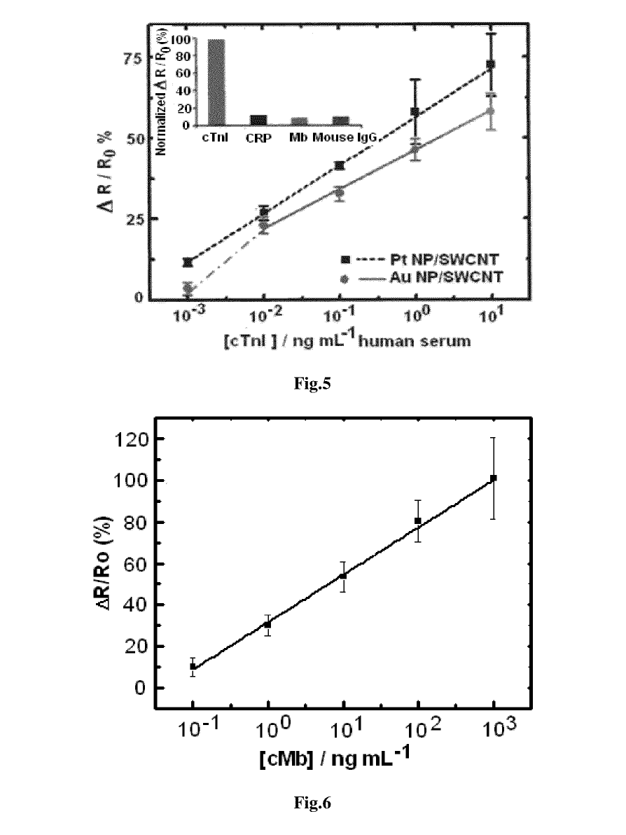

The sensing performance of the Ab-Mb-Pt(MPA)/SWCNTs hybrid device was investigated by exposing it to the different concentrations of human cardiac myoglobin antigen (Ag-Mb) in PBS (pH 7.4). A calibration curve was plotted between the normalized resistance change [(R−R₀)/R₀, where R₀ and R is the resistance of the device measured before and after exposure to human cardiac myoglobin antigen (Ag-Mb) in PBS, respectively] and logarithmic concentration of the target Ag-Mb. The resistance of the device was calculated by taking the reciprocal of the slope of the I-V plot between −0.5 and 0.5 V. The device was incubated with each 2 μL aliquot of different concentrations of Ag-Mb i.e. 0.1 ng mL⁻¹, 1.0 ng mL⁻¹, 10.0 ng mL⁻¹, 100 ng mL⁻¹ and 1000 ng mL⁻¹ at an interval 1 min, at 25° C. and rinsed thoroughly with double distilled water and dried under N₂ gas flow after each successive measurement I-V characteristics. The device exhibited a linear response to the target human cardiac myoglobin antigen, over the concentration range of 0.03 ng mL⁻¹ to 1000 ng mL⁻¹ with sensitivity (slope of the calibration curve) of 22.8% per decade. The detection limit of the device has been calculated by using the formula 3σ/m, where σ is the standard deviation of the blank and m is the slope of the calibration curve and is found to be 0.03 ng mL⁻¹ (FIG. 6).

### Example 15

### Inter and Intra Device Sensing Performance Towards the Target Myoglobin

The device sensing performance was investigated with three different chemiresistive devices prepared at different time. The results showed 8% intra and 12% inter-device relative standard deviation (RSD) in resistance upon exposure to a fixed concentration of 10 ng mL⁻¹ Ag-Mb.

### Example 16

### Selectivity of Device Towards Target Ag-Mb

Selectivity of device towards the target cardiac myoglobin, Ag-Mb, was investigated by exposing it with other cardiac specific biomarkers i.e. 100 ng mL⁻¹ C-reactive protein (CRP) and 100 ng mL⁻¹ non-specific protein antigen (mouse Immunoglobulin; IgG). The change in the normalized response of the device for CRP and IgG with respect to target Ag-Mb was found to be insignificant.

### Advantages of the Present Invention

The present invention provides a chemiresistive biosensor which is useful for the wide range of cardiac biomarkers cTnI and Mb detection.
The present invention provides a chemiresistive biosensor which requires easier operation with a small sample volume (about 2 μL) without additional reagents (label free)
The present invention provides a chemiresistive biosensor that requires less instrumental cost (a simple commercially available battery operated handheld multimeter that can measure pA to μA current).
The present invention provides a chemiresistive biosensor with significantly higher sensitivity due to ability of measuring very low electrical signals.
The chemiresistive bio sensor device provides a quick detection of myoglobin i.e. within about 1 min than conventional ELISA and spectroscopic techniques.
The chemiresistive biosensor device is sensitive to the lowest 0.001 ng mL⁻¹ cTnI detection in human serum.
The chemiresistive biosensor device has a lowest detection limit of 0.03 ng mL⁻¹ myoglobin in PBS

We claim:

1. A label free chemiresistive biosensor device for quantitative detection of human cardiac biomarker, wherein cardiac biomarkers are selected from human cardiac troponin I (cTnI) and myoglobin(Mb), said biosensor device comprising aligned carbon nanotubes channel, bridging an interspaced gap between microfabricated gold source and drain electrode over silicon dioxide coated silicon wafer having gold electrode surface passivated with thiol molecules and the carbon nanotubes channel being functionalized with capped platinum nanoparticles through a bilinker 1-pyrenemethylamine hydrochloride and said platinum nanoparticles being covalently immobilized with cardiac biomarker specific protein antibody anti-cTnI (Ab-cTnI) or anti-Mb (Ab-Mb) and blocking non-specific binding sites with a blocking reagent.

2. The device as claimed in claim 1, wherein the carbon nanotube is single-walled carbon nanotubes (SWCNTs) having 80% p-type semiconducting characteristics.

3. The device as claimed in claim 1, wherein the interspaced gap between the microfabricated gold source and drain electrodes is in the range of 1-5 μm.

4. The device as claimed in claim 1, wherein the platinum nanoparticles is capped with molecules having functional groups selected from the group consisting of carboxyl, amino and hydroxyl.

5. The device as claimed in claim 4, wherein the platinum nanoparticles is capped with mercaptopropionic acid.

6. The device as claimed in claim 1, wherein the size of the platinum nanoparticles is in the range of 2-20 nm.

7. The device as claimed in claim 6, wherein the size of the platinum nanoparticles in the range of 2-10 nm.

8. The device as claimed in claim 1, wherein the cardiac biomarker used is selected from cTnI and Mb. wherein the biosensor device is selective and sensitive to human cardiac cTnI in the range of 0.001 ng to 10.0 ng mL⁻¹ in normal human serum.

9. The device as claimed in claim 1, wherein the biosensor device is selective and sensitive to cTnI with a sensitivity of about 11% to 75% change in resistance over the concentration range of 0.001 ng to 10.0 ng mL⁻¹ cTnI in human serum.

10. The device as claimed in claim 1 wherein the biosensor device is sensitive to myoglobin in the range of 0.03 ng to 1000 ng mL⁻¹ in phosphate buffer saline. wherein the lowest detection limit is 0.03 ng mL⁻¹ for myoglobin.

11. The device as claimed in claim 1, wherein the thiol molecules are selected from the group consisting of 6-mercapto-1-haxanol and ethane hexanol.

12. The device as claimed in claim 1, wherein the blocking reagent is selected from the group consisting of bovine serum albumin, ethylamine and Polyoxyethylene (20) sorbitan monolaurate.

13. A process for the preparation of a label free chemiresistive biosensor device comprising the steps of:
i) bridging interspaced gap in between microfabricated gold electrode prepared over the silicon dioxide coated silicon substrate by electrophoretically aligning the single walled-carbon nanotubes, followed by annealing at a temperature of 200-400° C. under reduced environment of a gaseous mixture of 5-10% hydrogen in nitrogen gas, washing with distilled water and drying under N₂ gas flow to obtain a aligned single walled carbon nanotubes device (SWCNTs),
ii) treating the aligned single walled carbon nanotubes, as obtained in step (i) with a bilinker 1-pyrenemethylamine hydrochloride having terminal amino group in solvent for a period of 1-3 h and passivating gold source and drain electrodes by treating the carbon nanotubes with thiol molecule in dimethyl formamide for a period of 1-5 h, followed by washing successively with dimethyl formamide and distilled water and dried under N₂ gas flow to obtain bilinker modified single walled-carbon nanotubes,
iii) functionalizing bilinker modified single walled-carbon nanotubes, as obtained in step (ii) with carboxyl capped platinum nanoparticles (PtNP) by exposing the modified SWCNT-device to an aqueous solution of metal nanoparticles containing N-(3-dimethylaminopropyl)-N-ethyl carbodiimide hydrochloride and N-hydroxy succinimide for 1-5 h, rinsing thoroughly with double distilled water and drying under N₂ gas flow to obtain the PtNPs/SWCNT hybrid device,
iv) immobilizing cardiac biomarker specific protein antibody on PtNPs/SWCNT hybrid device, as obtained in step (iii) by exposing the hybrid device to antibody solution in phosphate buffer saline, at pH 7.0-7.6, at a temperature of 4-10° C., for a period of 10-24 h, followed by washing with double distilled water and drying under N₂ gas flow and passivating the device with a blocking reagent to obtain the chemiresistive biosensor device.

14. The process as claimed in claim 13, wherein thiol molecule is selected from the group consisting of 6-mercapto-1-haxanol and ethane hexanol.

15. The process as claimed in claim 14, wherein solvent is selected from the group consisting of dimethyl formamide and water or mixture thereof

16. The process as claimed in claim 14, wherein blocking reagent is selected from the group consisting of bovine serum albumin, ethylamine and Polyoxyethylene (20) sorbitan monolaurate.
